SEQUENCE LISTING (52 sequences)

SEQ ID NO: 1 - DNA, 19 bp, synthetic construct
  misc_feature 1..19: note = Primer
  source 1..19: mol_type = other DNA
gatgatacggtacatggcc

SEQ ID NO: 2 - DNA, 17 bp, synthetic construct
  misc_feature 1..17: note = Primer
  source 1..17: mol_type = other DNA
cttgcgcatgaccatgt

SEQ ID NO: 3 - DNA, 22 bp, synthetic construct
  misc_feature 1..22: note = Primer
  source 1..22: mol_type = other DNA
asaaatgatccgatatatggct

SEQ ID NO: 4 - DNA, 17 bp, synthetic construct
  misc_feature 1..17: note = Primer
  source 1..17: mol_type = other DNA
ctcgtcctcgttgtgga

SEQ ID NO: 5 - DNA, 23 bp, synthetic construct
  misc_feature 1..23: note = Primer
  source 1..23: mol_type = other DNA
ctccytacattggagttaaatgg

SEQ ID NO: 6 - DNA, 19 bp, synthetic construct
  misc_feature 1..19: note = Primer
  source 1..19: mol_type = other DNA
cctactgcaataccgcaag

SEQ ID NO: 7 - DNA, 20 bp, synthetic construct
  misc_feature 1..20: note = Primer
  source 1..20: mol_type = other DNA
taactgtagactcggcttgg

SEQ ID NO: 8 - DNA, 22 bp, synthetic construct
  misc_feature 1..22: note = Primer
  source 1..22: mol_type = other DNA
ccctttatacgctcaagcaata

SEQ ID NO: 9 - DNA, 20 bp, synthetic construct
  misc_feature 1..20: note = Primer
  source 1..20: mol_type = other DNA
cggtcgaagacaaagacaag

SEQ ID NO: 10 - DNA, 20 bp, synthetic construct
  misc_feature 1..20: note = Primer
  source 1..20: mol_type = other DNA
gttcatcccacgctttaact

SEQ ID NO: 11 - DNA, 18 bp, synthetic construct
  misc_feature 1..18: note = Primer
  source 1..18: mol_type = other DNA
tgccgcgatatattccgc

SEQ ID NO: 12 - DNA, 19 bp, synthetic construct
  misc_feature 1..19: note = Primer
  source 1..19: mol_type = other DNA
gtttggatgcttttggcct

SEQ ID NO: 13 - DNA, 22 bp, synthetic construct
  misc_feature 1..22: note = Primer
  source 1..22: mol_type = other DNA
atcaacgtcaaytacacacttc

SEQ ID NO: 14 - DNA, 22 bp, synthetic construct
  misc_feature 1..22: note = Primer
  source 1..22: mol_type = other DNA
tagagatccttacgaacatcga

SEQ ID NO: 15 - DNA, 22 bp, synthetic construct
  misc_feature 1..22: note = Primer
  source 1..22: mol_type = other DNA
cctttcaacaaaagggtatagc

SEQ ID NO: 16 - DNA, 18 bp, synthetic construct
  misc_feature 1..18: note = Primer
  source 1..18: mol_type = other DNA
gtatgtggacatccccca

SEQ ID NO: 17 - DNA, 23 bp, synthetic construct
  misc_feature 1..23: note = Primer
  source 1..23: mol_type = other DNA
cacattttcacttgtttgaagtg

SEQ ID NO: 18 - DNA, 20 bp, synthetic construct
  misc_feature 1..20: note = Primer
  source 1..20: mol_type = other DNA
tcgtccataagcaactttgc

SEQ ID NO: 19 - DNA, 24 bp, synthetic construct
  misc_feature 1..24: note = Primer
  source 1..24: mol_type = other DNA
gttgaccatatgcagtgttatttg

SEQ ID NO: 20 - DNA, 22 bp, synthetic construct
  misc_feature 1..22: note = Primer
  source 1..22: mol_type = other DNA
tatcacccattgttagtgttcc

SEQ ID NO: 21 - DNA, 22 bp, synthetic construct
  misc_feature 1..22: note = Primer
  source 1..22: mol_type = other DNA
accaaagattacaagatgctcc

SEQ ID NO: 22 - DNA, 24 bp, synthetic construct
  misc_feature 1..24: note = Primer
  source 1..24: mol_type = other DNA
cagttaaatcaacgaatgawccag

SEQ ID NO: 23 - DNA, 18 bp, synthetic construct
  misc_feature 1..18: note = Primer
  source 1..18: mol_type = other DNA
ttctccctcaaaccgctg

SEQ ID NO: 24 - DNA, 19 bp, synthetic construct
  misc_feature 1..19: note = Primer
  source 1..19: mol_type = other DNA
ccgcaagcaatgtttttgg

SEQ ID NO: 25 - DNA, 20 bp, synthetic construct
  misc_feature 1..20: note = Primer
  source 1..20: mol_type = other DNA
ggatcagattgcagctactg

SEQ ID NO: 26 - DNA, 22 bp, synthetic construct
  misc_feature 1..22: note = Primer
  source 1..22: mol_type = other DNA
gaagtccaaatcaagaccaaac

SEQ ID NO: 27 - DNA, 18 bp, synthetic construct
  misc_feature 1..18: note = Primer
  source 1..18: mol_type = other DNA
ggagtgcatagtaggtga

SEQ ID NO: 28 - DNA, 19 bp, synthetic construct
  misc_feature 1..19: note = Primer
  source 1..19: mol_type = other DNA
gtaacaacggataactcta

SEQ ID NO: 29 - DNA, 19 bp, synthetic construct
  misc_feature 1..19: note = Primer
  source 1..19: mol_type = other DNA
aagtgtgacctgaagatgt

SEQ ID NO: 30 - DNA, 19 bp, synthetic construct
  misc_feature 1..19: note = Primer
  source 1..19: mol_type = other DNA
aagtgtgacctgaagatgt

SEQ ID NO: 31 - DNA, 19 bp, synthetic construct
  misc_feature 1..19: note = Primer
  source 1..19: mol_type = other DNA
ctaaggctaactgacaatg

SEQ ID NO: 32 - DNA, 18 bp, synthetic construct
  misc_feature 1..18: note = Primer
  source 1..18: mol_type = other DNA
tccaccagataccaagtc

SEQ ID NO: 33 - DNA, 19 bp, synthetic construct
  misc_feature 1..19: note = Primer
  source 1..19: mol_type = other DNA
atcgtgagtcatccatagt

SEQ ID NO: 34 - DNA, 19 bp, synthetic construct
  misc_feature 1..19: note = Primer
  source 1..19: mol_type = other DNA
tactgactaacgagacgag

SEQ ID NO: 35 - DNA, 20 bp, synthetic construct
  misc_feature 1..20: note = Primer
  source 1..20: mol_type = other DNA
atgtatccccagactgtaac

SEQ ID NO: 36 - DNA, 19 bp, synthetic construct
  misc_feature 1..19: note = Primer
  source 1..19: mol_type = other DNA
aacatcaggcaatcgtaag

SEQ ID NO: 37 - DNA, 19 bp, synthetic construct
  misc_feature 1..19: note = Primer
  source 1..19: mol_type = other DNA
gctgttcggtttatgtcac

SEQ ID NO: 38 - DNA, 19 bp, synthetic construct
  misc_feature 1..19: note = Primer
  source 1..19: mol_type = other DNA
tgagatttagcgtagattt

SEQ ID NO: 39 - DNA, 18 bp, synthetic construct
  misc_feature 1..18: note = Primer
  source 1..18: mol_type = other DNA
gtaagcaatagcaccaaa

SEQ ID NO: 40 - DNA, 18 bp, synthetic construct
  misc_feature 1..18: note = Probe
  source 1..18: mol_type = other DNA
tcacctactatgcactcc

SEQ ID NO: 41 - DNA, 19 bp, synthetic construct
  misc_feature 1..19: note = Probe
  source 1..19: mol_type = other DNA
tagagttatccgttgttac

SEQ ID NO: 42 - DNA, 19 bp, synthetic construct
  misc_feature 1..19: note = Probe
  source 1..19: mol_type = other DNA
acatcttcaggtcacactt

SEQ ID NO: 43 - DNA, 19 bp, synthetic construct
  misc_feature 1..19: note = Probe
  source 1..19: mol_type = other DNA
cattgtcagttagccttag

SEQ ID NO: 44 - DNA, 18 bp, synthetic construct
  misc_feature 1..18: note = Probe
  source 1..18: mol_type = other DNA
gacttggtatctggtgga

SEQ ID NO: 45 - DNA, 19 bp, synthetic construct
  misc_feature 1..19: note = Probe
  source 1..19: mol_type = other DNA
actatggatgactcacgat

SEQ ID NO: 46 - DNA, 19 bp, synthetic construct
  misc_feature 1..19: note = Probe
  source 1..19: mol_type = other DNA
ctcgtctcgttagtcagta

SEQ ID NO: 47 - DNA, 20 bp, synthetic construct
  misc_feature 1..20: note = Probe
  source 1..20: mol_type = other DNA
gttacagtctggggatacat

SEQ ID NO: 48 - DNA, 19 bp, synthetic construct
  misc_feature 1..19: note = Probe
  source 1..19: mol_type = other DNA
cttacgattgcctgatgtt

SEQ ID NO: 49 - DNA, 19 bp, synthetic construct
  misc_feature 1..19: note = Probe
  source 1..19: mol_type = other DNA
gtgacataaaccgaacagc

SEQ ID NO: 50 - DNA, 19 bp, synthetic construct
  misc_feature 1..19: note = Probe
  source 1..19: mol_type = other DNA
aaatctacgctaaatctca

SEQ ID NO: 51 - DNA, 18 bp, synthetic construct
  misc_feature 1..18: note = Probe
  source 1..18: mol_type = other DNA
tttggtgctattgcttac

SEQ ID NO: 52 - DNA, 20 bp, synthetic construct
  misc_feature 1..20: note = Probe
  source 1..20: mol_type = other DNA
cctattatgtattgacgatg

(19) United States
(12) Patent Application Publication
(10) Pub. No.: US 2025/0270664 A1
(43) Pub. Date: Aug. 28, 2025

(54) KIT FOR DETECTING PATHOGENS CAUSING SEXUALLY TRANSMITTED INFECTIONS AND METHOD FOR DETECTING PATHOGENS CAUSING SEXUALLY TRANSMITTED INFECTIONS USING SAME

(71) Applicant: PAXGENBIO CO., LTD., Gyeonggi-do (KR)

(72) Inventors: Yeeun SHIN, Gyeonggi-do (KR); Byoungoh HWANG, Gyeonggi-do (KR); Chanhyo LEE, Jeju-do (KR); Young Suk PARK, Gyeonggi-do (KR)

(21) Appl. No.: 18/858,045

(22) PCT Filed: Apr. 20, 2023
(86) PCT No.: PCT/KR2023/005348

§ 371 Date: Oct. 18, 2024

(30) Foreign Application Priority Data
Apr. 21, 2022 (KR) 10-2022-0049599

(51) Int. Cl. C12Q 1/70; C12Q 1/689

(52) U.S. Cl. CPC C12Q 1/701; C12Q 1/689; C12Q 2600/16; G01N 2800/26; G01N 2800/34

(57) ABSTRACT

The present invention relates to a kit for detecting sexually transmitted infection-causing pathogens selected from the group consisting of 12 sexually transmitted infection (STI)-causing pathogens, including Chlamydia trachomatis (CT), Neisseria gonorrhea (NG), Treponema pallidum (TP), Trichomonas vaginalis (TV), Ureaplasma urealyticum (UU), Ureaplasma parvum (UP), Mycoplasma genitalium (MG), Mycoplasma hominis (MH), Gardnerella vaginalis (GV), Candida albicans (CA), Herpes simplex virus 1 (HSV1), and Herpes simplex virus 2 (HSV2), and a method of detecting sexually transmitted infection-causing pathogens using the same.

### TECHNICAL FIELD

The present invention relates to a kit for detecting sexually transmitted infection-causing pathogens selected from the group consisting of 12 sexually transmitted infection (STI)-causing pathogens, including Chlamydia trachomatis (CT), Neisseria gonorrhea (NG), Treponema pallidum (TP), Trichomonas vaginalis (TV), Ureaplasma urealyticum (UU), Ureaplasma parvum (UP), Mycoplasma genitalium (MG), Mycoplasma hominis (MH), Gardnerella vaginalis (GV), Candida albicans (CA), Herpes simplex virus 1 (HSV1), and Herpes simplex virus 2 (HSV2), and a method of detecting sexually transmitted infection-causing pathogens using the same.

### BACKGROUND ART

Sexually transmitted infections (STIs) are mainly spread through sexual contact. Some sexually transmitted infections can be passed on to children during pregnancy, childbirth, or breastfeeding. Common symptoms of sexually transmitted infections include vaginal discharge, urethral discharge or burning in men, genital ulcers, and abdominal pain, and sometimes there are no symptoms of the disease. These sexually transmitted infections have a significant impact on sexual and reproductive health worldwide.

More than one million cases of sexually transmitted infections occur every year. In 2020, the WHO estimated the number of newly infected patients with one or more of the following four sexually transmitted infection-causing pathogens at 374 million: chlamydia (129 million), gonorrhea (82 million), syphilis (7.1 million), and trichomoniasis (156 million). In addition, in 2016, it was estimated that 490 million people were infected with genital HSV (herpes).

Meanwhile, sexually transmitted infections such as herpes, gonorrhea, or syphilis can increase HIV infection.

Mother-to-child transmission of sexually transmitted infections can result in stillbirth, neonatal death, low birth weight and prematurity, sepsis, pneumonia, neonatal conjunctivitis, and congenital malformations. In 2016, approximately one million pregnant women were infected with active syphilis, of which 350,000 had negative birth outcomes and 200,000 had stillbirths or neonatal deaths.

In addition, sexually transmitted infections such as gonorrhea and chlamydiosis are major causes of pelvic inflammatory disease and female infertility.

Currently, there are molecular diagnostic methods for diagnosing transmitted infections sexually using electrophoresis after conventional PCR or using real-time PCR. Representative companies that manufacture molecular diagnostic kits using conventional PCR-electrophoresis typically include Diogene Co., Ltd. and TCM Biosciences Inc., and companies that manufacture real-time PCR kits typically include Seegene Inc., Bioneer Corp., and Panagene Inc.

The conventional PCR-electrophoresis method has a complicated process, and the agarose gel analysis method for analyzing the results uses human vision, which may lead to errors in the results. In addition, real-time PCR disadvantageously requires an expensive detection device and uses a fluorescent probe as a detection agent, which increases the kit cost.

Under this technical background, the inventors of the present application have developed a kit that can detect 12 sexually transmitted infection-causing pathogens simultaneously, quickly, accurately, and qualitatively, and can detect sexually transmitted infection-causing pathogens using only trace amounts of sample genes, and a method of detecting infection with sexually transmitted infection-causing pathogens using the same, thereby completing the present invention.

### SUMMARY OF THE INVENTION

The present invention has been made keeping in mind the above-mentioned problems occurring in the prior art, and an object of the present invention is to provide a kit for detecting 12 sexually transmitted infection-causing pathogens, which can diagnose infection with 12 sexually transmitted infection-causing pathogens more accurately and simply than the prior art, a method for detecting sexually transmitted infection-causing pathogens, and a method of diagnosing infection with 12 sexually transmitted infection-causing pathogens using the same.

To achieve the above object, the present invention is directed to the use of (a) a primer containing: a complementary nucleic acid oligomer capable of binding to and amplifying a gene specific to a sexually transmitted infection-causing pathogen selected from the group consisting of Chlamydia trachomatis (CT), Neisseria gonorrhea (NG), Treponema pallidum (TP), Trichomonas vaginalis (TV), Ureaplasma urealyticum (UU), Ureaplasma parvum (UP), Mycoplasma genitalium (MG), Mycoplasma hominis(MH), Gardnerella vaginalis (GV), Candida albicans (CA), Herpes simplex virus 1 (HSV1), and Herpes simplex virus 2 (HSV2); and a nucleic acid oligomer non-complementary to the specific gene; and (b) a probe that binds complementary to the non-complementary nucleic acid oligomer, for detection of sexually transmitted infection-causing pathogens.

To achieve the above object, the present invention is directed to a kit for detecting sexually transmitted infection-causing pathogens, comprising: (a) a primer containing: a complementary nucleic acid oligomer capable of binding to and amplifying a gene specific to a sexually transmitted infection-causing pathogen selected from the group consisting of Chlamydia trachomatis (CT), Neisseria gonorrhea (NG), Treponema pallidum (TP), Trichomonas vaginalis (TV), Ureaplasma urealyticum (UU), Ureaplasma parvum (UP), Mycoplasma genitalium (MG), Mycoplasma hominis (MH), Gardnerella vaginalis (GV), Candida albicans (CA),

Herpes simplex virus 1 (HSV1), and Herpes simplex virus 2 (HSV2); and a nucleic acid oligomer non-complementary to the specific gene; and (b) a probe that binds complementary to the non-complementary nucleic acid oligomer.

The present invention is also directed to a method of detecting sexually transmitted infection-causing pathogens in vitro using the above kit.

The present invention is also directed to a method for detecting sexually transmitted infection-causing pathogens, comprising steps of: (a) reacting a nucleic acid extracted from a sample with a primer containing: a complementary nucleic acid oligomer capable of binding to and amplifying a gene specific to a sexually transmitted infection-causing pathogen selected from the group consisting of Chlamydia trachomatis (CT), Neisseria gonorrhea (NG), Treponema pallidum (TP), Trichomonas vaginalis (TV), Ureaplasma urealyticum (UU), Ureaplasma parvum (UP), Mycoplasma genitalium (MG), Mycoplasma hominis (MH), Gardnerella vaginalis (GV), Candida albicans (CA), Herpes simplex virus 1 (HSV1), and Herpes simplex virus 2 (HSV2); and a nucleic acid oligomer non-complementary to the gene specific to the sexually transmitted infection-causing pathogen; and (b) reacting the product of the reaction with a probe that binds complementary to the non-complementary nucleic acid oligomer.

### BRIEF DESCRIPTION OF DRAWINGS

FIG. 1 shows the process for preparing a primer and a probe, which are contained in a kit according to an embodiment of the present invention.

FIG. 2 is a schematic diagram showing a universal lateral flow assay (ULFA) reaction for confirming an amplification product in a kit according to an embodiment of the present invention.

FIG. 3 illustrates the positions of probes immobilized on the kit according to an embodiment of the present invention and the types of sexually transmitted infection pathogens applied thereto.

FIG. 4 shows the results of testing 12 sexually transmitted infection-causing pathogens via negative or positive criteria using a kit according to an embodiment of the present invention.

FIG. 5 shows the results of amplifying and detecting sexually transmitted infection-causing pathogens using a kit according to an embodiment of the present invention.

### DETAILED DESCRIPTION AND PREFERRED EMBODIMENTS OF THE INVENTION

Unless otherwise defined, all technical and scientific terms used in the present specification have the same meanings as commonly understood by those skilled in the art to which the present disclosure pertains. In general, the nomenclature used in the present specification is well known and commonly used in the art.

The inventors of the present application have prepared an STI PaxView STI12 MPCR-ULFA kit comprising: a primer containing a complementary nucleic acid oligomer that binds to a gene specific to a sexually transmitted infection-causing pathogen selected from the group consisting of Chlamydia trachomatis (CT), Neisseria gonorrhea (NG), Treponema pallidum (TP), Trichomonas vaginalis (TV), Ureaplasma urealyticum (UU), Ureaplasma parvum (UP), Mycoplasma genitalium (MG), Mycoplasma hominis (MH), Gardnerella vaginalis (GV), Candida albicans (CA), Herpes simplex virus 1 (HSV1), and Herpes simplex virus 2 (HSV2); and a nucleic acid oligomer non-complementary to the gene specific to the sexually infection-causing pathogen, and a nucleic acid oligomer non-complementary to the gene specific to the sexually transmitted infection-causing pathogen selected from the group consisting of HSV1, HSV2, CT, NG, TP, TV, UU, UP, MG, MH, CA, and GV; and a membrane having immobilized thereon a probe that binds complementary to the non-complementary nucleic acid oligomer. This kit is a molecular diagnostic kit that quickly, accurately and qualitatively identifies the genotypes of 12 sexually transmitted infection-causing pathogens, and it has been found that, when a gene extracted from a human cervical smear is used as a template, amplified and loaded in the ULFA device and the nanogold particles appear in the form of bands, HSV1, HSV2, CT, NG, TP, TV, UU, UP, MG, MH, CA, and GV infections can be diagnosed.

Therefore, in one aspect, the present invention is directed to a kit for detecting sexually transmitted infection-causing pathogens, comprising: (a) a primer containing: a complementary nucleic acid oligomer capable of binding to and amplifying a gene specific to a sexually transmitted infection-causing pathogen selected from the group consisting of Chlamydia trachomatis (CT), Neisseria gonorrhea (NG), Treponema pallidum (TP), Trichomonas vaginalis (TV), Ureaplasma urealyticum (UU), Ureaplasma parvum (UP), Mycoplasma genitalium (MG), Mycoplasma hominis (MH), Gardnerella vaginalis (GV), Candida albicans (CA), Herpes simplex virus 1 (HSV1), and Herpes simplex virus 2 (HSV2); and a nucleic acid oligomer non-complementary to the specific gene; and (b) a probe that binds complementary to the non-complementary nucleic acid oligomer.

The kit according to the present invention may overcome the shortcomings of other companies' kits for detecting sexually transmitted infection-causing pathogens and maximize their advantages, thereby occupying the market and providing a new platform for diagnosing sexually transmitted infection-related diseases.

Among molecular diagnostic products, products that use polymerase chain reaction (PCR) technology can be broadly classified into two categories. One is conventional PCR, which extracts nucleic acid from a sample, amplifies the target nucleic acid, and then checks the result by electrophoresis. Using this method, an inexpensive kit is available, but electrophoresis requires a lot of labor and time. Real-time PCR, a technique used instead of electrophoresis, can monitor pathogen nucleic acid amplification in real time, but the kit is expensive.

Accordingly, there is a need to develop a molecular diagnostic product which is inexpensive while ensuring user convenience. Thus, the inventors of the present application have developed an in vitro diagnostic kit for qualitative analysis.

The kit according to the present invention comprises a primer containing a complementary nucleic acid oligomer that binds to a gene specific to one selected from 12 sexually transmitted infection pathogen-specific genes, and a nucleic acid oligomer non-complementary to the specific gene, wherein the primer contains a gene specific to at least one sexually transmitted infection pathogen selected from the group consisting of HSV1, HSV2, CT, NG, TP, TV, UU, UP, MG, MH, CA, and GV, and a nucleic acid oligomer containing nucleotides non-complementary thereto.

The gene specific to the sexually transmitted infection pathogen may be one or more or two or more genes that are specific to sexually transmitted infection pathogens. For example, the gene may be selected from the group consisting of the ompA gene and cryptic plasmid of Chlamydia trachomatis, and may be the ompA gene and/or cryptic plasmid of CT.

The gene specific to the sexually transmitted infection pathogen may be, for example, the dcm/dcr gene of Neisseria gonorrhoeae. For Treponema pallidum, the specific gene may be, for example, bamA gene. For Trichomonas vaginalis, the specific gene may be, for example, actin gene. For Ureaplasma urealyticum, the specific gene may be, for example, ureG gene. For Ureaplasma parvum, the specific gene may be, for example, ureB gene. For Mycoplasma genitalium, the specific gene may be, for example, mgpA gene. For Mycoplasma hominis, the specific gene may be, for example, gap gene. For Candida albicans, the specific gene may be, for example, 16SrRNA. For Gardnerella vaginalis, the specific gene may be, for example, cpn60 gene. For Herpes simplex virus 1, the specific gene may be, for example, gB gene. For Herpes simplex virus 2, the specific gene may be, for example, gB gene.

In the present specification, “sexually transmitted infection” refers to infections caused by organisms that are transmitted between humans through sexual activity and close contact, and “sexually transmitted infection-causing pathogens” includes microorganisms, yeast, or viruses that are transmitted between humans through sexual activity and close contact. Sexually transmitted infection-causing pathogens detectable according to the present invention are Chlamydia trachomatis (CT), Neisseria gonorrhea (NG), Treponema pallidum (TP), Trichomonas vaginalis (TV), Ureaplasma urealyticum (UU), Ureaplasma parvum (UP), Mycoplasma genitalium (MG), Mycoplasma hominis (MH), Gardnerella vaginalis (GV), Candida albicans (CA), Herpes simplex virus 1 (HSV1), and Herpes simplex virus 2 (HSV2).

Accordingly, the primer may contain a complementary nucleic acid oligomer that can specifically bind to and amplify a gene specific to each of sexually transmitted infection pathogens, and a nucleic acid oligomer containing nucleotides non-complementary to a gene present in each of the sexually transmitted infection pathogens. According to the present invention, it is possible to simultaneously identify and diagnose infections with 12 genotypes of sexually transmitted infection pathogens.

In the present specification, the “primer” refers to a single-stranded oligonucleotide that can act as a point of initiation for template-directed DNA synthesis under appropriate conditions (i.e., four different nucleoside triphosphates and a polymerization enzyme) in an appropriate buffer and at a suitable temperature.

The primer according to the present invention may contain a fragment of a gene specific to each of 12 STIs. The fragment may refer to any region of a gene specific to each of 12 STIs. The fragment may have any length. For example, the fragment may refer to a region having a length of 5 bp to 50 bp, specifically 10 bp to 30 bp, which is specific to a target gene or any region thereof.

In the present specification, “complementary binding” means that the primer hybridizes with the corresponding nucleic acid strand under the conditions for performing a polymerization reaction, and a duplex structure may be formed by complementary binding. Complementary binding can be formed even if the complementarity between paired nucleotide sequences forms a Watson-Crick pair or some non-Watson-Crick base pairs exist.

The primer includes a forward primer and may contain, for example, a complementary nucleic acid oligomer that specifically binds to a gene to each of 12 sexually transmitted infection pathogens. When the forward sexually transmitted infection pathogen-specific gene is used as a template, it is possible to specifically amplify a sequence complementary to the sexually transmitted infection pathogen-specific gene by binding to the complementary strand.

The primer contains a nucleic acid oligomer containing nucleotides non-complementary to the sexually transmitted infection pathogen-specific gene or fragment thereof. When amplifying a sexually transmitted infection pathogen-specific gene as a target by PCR, the primer may contain a nucleic acid oligomer containing nucleotides non-complementary to the sexually transmitted infection pathogen-specific gene, that is, a gene unrelated to the STI-specific gene, which has a length of about 20-60 bp, specifically 20-40 bp, preferably about 20-30 bp. By reacting this nucleic acid oligomer with a complementary probe synthesized on a membrane, it is possible to easily check the result, that is, whether the nucleic acid has been amplified.

The primer may further include a primer containing a tag. The primer containing the tag is a reverse primer, and when the sexually transmitted infection pathogen-specific gene is used as a template, the reverse primer can amplify the template by complementary binding to the template strand.

For example, the primer may contain a complementary nucleic acid oligomer comprising a sequence selected from the group consisting of the sequences of SEQ ID NO: 1 to SEQ ID NO: 26.

Specifically, the primer may comprise:
a complementary nucleic acid oligomer comprising the sequences of SEQ ID NO: 1 and SEQ ID NO: 2, which binds to the HSV1-specific gene;
a complementary nucleic acid oligomer comprising the sequences of SEQ ID NO: 3 and SEQ ID NO: 4, which binds to the HSV2-specific gene;
a complementary nucleic acid oligomer comprising a sequence selected from the group consisting of SEQ ID NO: 5 to SEQ ID NO: 8, which binds to the CT-specific gene;
a complementary nucleic acid oligomer the sequences of SEQ ID NO: 9 and SEQ ID NO: 10, which binds to the NG-specific gene;
a complementary nucleic acid oligomer the sequences of SEQ ID NO: 11 and SEQ ID NO: 12, which binds to the TP-specific gene;
a complementary nucleic acid oligomer comprising the sequences of SEQ ID NO: 13 and SEQ ID NO: 14, which binds to the TV-specific gene;
a complementary nucleic acid oligomer comprising the sequences of SEQ ID NO: 15 and SEQ ID NO: 16, which binds to the UU-specific gene;
a complementary nucleic acid oligomer comprising the sequences of SEQ ID NO: 17 and SEQ ID NO: 18, which binds to the UP-specific gene;
a complementary nucleic acid oligomer comprising the sequences of SEQ ID NO: 19 and SEQ ID NO: 20, which binds to the MG-specific gene;
a complementary nucleic acid oligomer comprising the sequences of SEQ ID NO: 21 and SEQ ID NO: 22, which binds to the MH-specific gene;
a complementary nucleic acid oligomer comprising the sequences of SEQ ID NO: 23 and SEQ ID NO: 24, which binds to the CA-specific gene; or
a complementary nucleic acid oligomer comprising the sequences of SEQ ID NO: 25 and SEQ ID NO: 26, which binds to the GV-specific gene.

When primers containing nucleic acid oligomers complementary to multiple sexually transmitted infection- causing pathogen genotypes are used in one panel of the kit, wherein panel may include the primers of SEQ ID NOS: 1 to 26.

For reaction with complementary probes synthesized on a membrane, a non-nucleotide spacer may be further included between nucleic acid oligomers non-complementary to 12 genotypes of sexually transmitted infection-causing pathogen-specific genes or fragments thereof and nucleotides for binding to the complementary probes synthesized on the membrane.

For example, the non-complementary nucleic acid oligomer may be selected from the group consisting of sequences of SEQ ID NOs: 27 to 39. Specifically, the non-complementary nucleic acid oligomer may comprise: nucleic acid oligomer comprising the sequence of SEQ ID NO: 27, which is non-complementary to the HSV1-specific gene;
a nucleic acid oligomer comprising the sequence of SEQ ID NO: 28, which is non-complementary to the HSV2-specific gene;
a nucleic acid oligomer comprising the sequence of SEQ ID NO: 29 or SEQ ID NO: 30, which is non-complementary to the CT-specific gene;
a nucleic acid oligomer comprising the sequence of SEQ ID NO: 31, which is non-complementary to the NG-specific gene;
a nucleic acid oligomer comprising the sequence of SEQ ID NO: 32, which is non-complementary to the TP-specific gene;
a nucleic acid oligomer comprising the sequence of SEQ ID NO: 33, which is non-complementary to the TV-specific gene;
a nucleic acid oligomer comprising the sequence of SEQ ID NO: 34, which is non-complementary to the UU-specific gene;
a nucleic acid oligomer comprising the sequence of SEQ ID NO: 35, which is non-complementary to the UP-specific gene;
a nucleic acid oligomer comprising the sequence of SEQ ID NO: 36, which is non-complementary to the MG-specific gene;
a nucleic acid oligomer comprising the sequence of SEQ ID NO: 37, which is non-complementary to the MH-specific gene;
a nucleic acid oligomer comprising the sequence of SEQ ID NO: 38, which is non-complementary to the CA-specific gene; or
a nucleic acid oligomer comprising the sequence of SEQ ID NO: 39, which is non-complementary to the GV-specific gene.

The non-nucleotide spacer may be a C3, C6, C9 or C12 aliphatic spacer, wherein the number after C indicates the number of carbon atoms in the non-nucleotide spacer structure. This non-nucleotide spacer may be an alkyl, alkenyl, or alkynyl group.

In one embodiment, when a non-nucleotide spacer, for example, a C3 spacer (propyl spacer), is located between nucleotide sequence regions non-complementary to sexually transmitted infection-causing pathogen-specific genes or fragments thereof and nucleotides for binding to complementary probes synthesized on a membrane and when the gene is amplified using Taq polymerase, complementary amplification may be prevented from invading the nucleotide sequence region for binding to the probe, so that a partial single-stranded nucleic acid product can be obtained, and the nucleotide sequence region can bind to the probe on the membrane.

In one embodiment, the tag contained in the primer may be biotin, Cy5, Cy3, FITC, EDANS (5-(2′-aminoethyl) amino-1-naphthalene sulfate), tetramethylrhodamine (TMR), tetramethylrhodamine isocyanate (TMRITC), x-rhodamine, DIG, or an antibody, or nanoparticles bound thereto, without being limited thereto.

Here, by reacting with a binding agent that elicits a chromogenic signal of the tag, it is possible to confirm a chromogenic or fluorescent signal and detect amplification of the target nucleic acid. In this case, the binding agent may be streptavidin, without being limited thereto.

In a specific embodiment, when amplification is performed using a reverse primer having biotin attached thereto, the biotin can bind to streptavidin on the membrane. According to the tendency of particles (e.g., nanoparticles) conjugated to the biotin, target nucleic acid amplification can be confirmed by a chromogenic signal in the case of gold particles, or can be confirmed by fluorescent signals having various wavelengths. Thus, depending on the kind of probe attached to the membrane, amplification of several to several tens of target nucleic acids can be detected.

The kit according to the present invention comprises (b) a membrane having immobilized thereon a probe that binds complementary to the nucleic acid oligomer. Accordingly, when the product amplified by the primer is injected into the lateral side of the membrane, the amplified product may move and a complementary hybridization reaction between the probe immobilized on the membrane and the nucleic acid oligomer contained in the amplified product may occur.

The “membrane” may be formed from any materials. For example, the membrane may be formed from natural, synthetic, or naturally occurring materials modified by synthesis, such as polysaccharides (e.g., cellulose materials such as paper and cellulose derivatives, such as cellulose acetate and nitrocellulose); polyether sulfone; polyethylene; nylon; polyvinylidene fluoride (PVDF); polyester; polypropylene; silica; inorganic materials, such as deactivated alumina, diatomaceous earth, MgSO₄, or other inorganic finely divided material uniformly dispersed in a porous polymer matrix, with polymers such as vinyl chloride, vinyl chloride-propylene copolymer, and vinyl chloride-vinyl acetate copolymer; cloth, both naturally occurring (e.g., cotton) and synthetic (e.g., nylon or rayon); porous gels, such as silica gel, agarose, dextran, and gelatin; polymeric films, such as polyacrylamide; and so forth. Preferably, the membrane may include polymeric materials such as nitrocellulose, polyethersulfone, polyethylene, nylon, polyvinylidene fluoride, polyester, and polypropylene.

The primers according to the present invention may be used in multiplex-PCR, making it possible to diagnose infection by simultaneously detecting 12 sexually transmitted infection-causing pathogens. That is, when multiplex PCR is performed using primers with several to dozens of different oligomer sequences, and then the PCR amplification products are allowed to react with oligomeric probes complementary to the oligomer sequences, it is possible to identify several to dozens of amplification products using a single lateral flow membrane.

Thus, as a lateral flow membrane may be used commonly in various multiplex PCR assays, large amounts of results from a lateral flow membrane can be produced. This indicates that various amplification products can be identified using a single lateral flow membrane. That is, as amplification products can be identified using the same membrane regardless of the kind of PCR amplification product, production cost reduction and quality control can be easily achieved, thereby increasing productivity.

The probe that binds complementary to the nucleic acid oligomer may comprise, for example, one or more sequences selected from the group consisting of SEQ ID NOS: 27 to 39.

In some embodiments, the probe may additionally comprises a nucleic acid oligomer for immobilization on the membrane, and the probe may further comprise a 20-60 bp, specifically 20-40 bp, oligomer having repeated nucleotide sequences.

In some embodiments, the kit according to the present invention may optionally additionally comprise an internal control primer. This internal control primer is used to avoid false negative results, that is, to check whether the PCR reaction has been performed properly, and it is possible to select any gene that is normally expressed regardless of the presence or absence of STIs in the sample.

The kit according to the present invention may optionally comprise reagents required for performing nucleic acid amplification PCR reaction, such as polymerase, buffer, and deoxyribonucleotide-5-triphosphate. The kit according to the present invention may also further comprise various polynucleotide molecules, and various buffers and reagents.

The optimal amount of a reagent, a buffer or a reactant used for a specific reaction in the kit may be determined by those skilled in the art, and the kit may be manufactured as a separate package or compartment containing (i) the primer (forward) containing the complementary nucleic acid oligomer that binds complementary to the above-mentioned STI-specific gene and the nucleic acid oligomer containing nucleotides non-complementary thereto, and/or (ii) the primer (reverse) containing the tag, and the membrane having immobilized thereon the probe.

In another aspect, the present invention is directed to a method of detecting STIs in vitro using the above-described kit.

The present invention is also directed to a method of providing information for detection of sexually transmitted infection-causing pathogens, comprising steps of: (a) reacting a nucleic acid extracted from a sample with a primer containing: a complementary nucleic acid oligomer capable of binding to and amplifying a gene specific to a sexually transmitted infection-causing pathogen selected from the group consisting of Chlamydia trachomatis (CT), Neisseria gonorrhea (NG), Treponema pallidum (TP), Trichomonas vaginalis (TV), Ureaplasma urealyticum (UU), Ureaplasma parvum (UP), Mycoplasma genitalium (MG), Mycoplasma hominis (MH), Gardnerella vaginalis (GV), Candida albicans (CA), Herpes simplex virus 1 (HSV1), and Herpes simplex virus 2 (HSV2); and a nucleic acid oligomer non-complementary to the gene specific to the sexually transmitted infection-causing pathogen; and (b) reacting the product of the reaction with a probe that binds complementary to the non-complementary nucleic acid oligomer.

The present invention is also directed to a method for detecting sexually transmitted infection-causing pathogens, comprising steps of: (a) reacting a nucleic acid extracted from a sample with a primer containing: a complementary nucleic acid oligomer capable of binding to and amplifying a gene specific to a sexually transmitted infection-causing pathogen selected from the group consisting of Chlamydia trachomatis (CT), Neisseria gonorrhea (NG), Treponema pallidum (TP), Trichomonas vaginalis (TV), Ureaplasma urealyticum (UU), Ureaplasma parvum (UP), Mycoplasma genitalium (MG), Mycoplasma hominis (MH), Gardnerella vaginalis (GV), Candida albicans (CA), Herpes simplex virus 1 (HSV1), and Herpes simplex virus 2 (HSV2); and a nucleic acid oligomer non-complementary to the gene specific to the sexually transmitted infection-causing pathogen; and (b) reacting the product of the reaction with a probe that binds complementary to the non-complementary nucleic acid oligomer.

The description of the kit and related configurations used in performing the method according to the present invention may be equally applied to the method.

In one embodiment, the sample may include a wide range of biological fluids obtained from patients suspected of having sexually transmitted infection pathogens, or individuals to be diagnosed, or bodily fluids, cell lines, tissue cultures, etc. derived from individuals. Examples of the sample include, but are not limited thereto, cervical and vaginal swabs, cervical tissue, tissue of the male genitalia, urine, anus, rectum, pharynx, oral cavity, and head and neck.

The method may further comprise a step of extracting nucleic acid from the sample, wherein the extraction of nucleic acid may be performed using, for example, various commercially available kits or extraction reagents.

### EXAMPLES

Hereinafter, the present invention will be described in more detail by way of examples. These examples are only for illustrating the present invention, and it will be apparent to those skilled in the art that the scope of the present invention is not to be construed as being limited by these examples.

### 1. Preparation of Kit

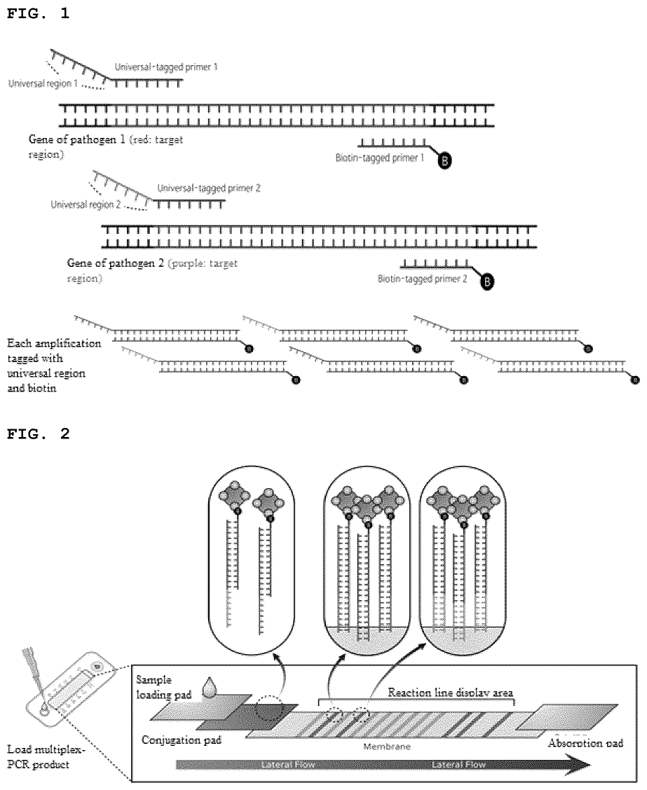

The kit according to the present invention (hereinafter also referred to as PaxView STI12 MPCR-ULFA kit) includes two kits. One is a kit for amplifying nucleic acid (hereinafter referred to as Component 1. STI12 MPCR kit), and the other is a kit for confirming the amplification product (hereinafter referred to Component 2. PaxView STI12 MPCR kit). The primers contained in the primer mixture that is a component of the PaxView STI12 MPCR-ULFA kit include regions for 12 types of STIs and a region for internal control, wherein the forward primer (sense) is tagged with a universal region, a non-complementary nucleic acid oligomer, and the reverse primer (antisense) is tagged with a biotin sequence (FIG. 1).

After PCR, when the amplification product is loaded into the ULFA device of the PaxView ULFA kit, it binds to the gold-streptavidin conjugate (gold-streptavidin conjugate=gold-SV, present on a pad made of glass fiber), and then binds to a probe (probe=oligomer complementary to the universal region) adsorbed on the nitrocellulose (NC) membrane, thereby developing color at the display site of each pathogen. The principle of color development is based on the reaction between biotin tagging each primer and streptavidin, and may be used to confirm DNA reactions in addition to the widely used antigen-antibody reaction. Since streptavidin, similar to avidin, can form a complex with biotin, visible color development occurs due to gold particles conjugated to biotin (FIG. 2).

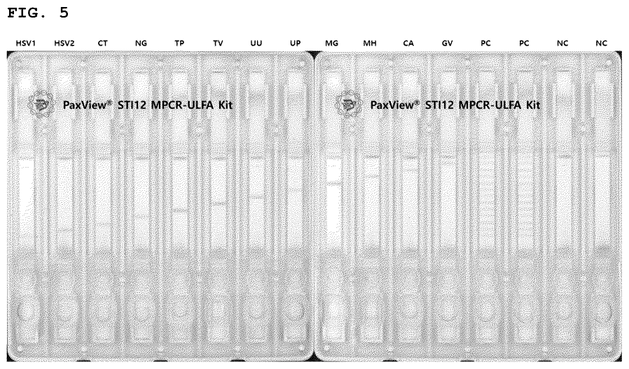

After color development, the ULFA device is read with the naked eye. Whether the results are negative or positive is determined according to the criteria described in the kit's usage instructions, and since the location of the coloring line varies depending on the type of STI, it can be confirmed that the expected band pattern and the actual band pattern match (FIGS. 4 and 5).

The STI diagnostic kit according to the present invention is a new platform for STI diagnosis. The kit is a rapid and accurate qualitative molecular diagnostic kit that can amplify the template gene extracted from the sample, and when the nano-gold particles in the ULFA device appears in the form of bands after loading of the PCR product, infection with 12 types of STIs can be diagnosed.

### 2. STI Diagnosis

### (1) Preparation of Mixture

1) PCR master mix was prepared as follows.

| | | |
|---|---|---|
| Composition | Quantity of 1 test | Quantity of 96 tests |

| | | | | |
|---|---|---|---|---|
| 2X PCR premix | 10 | μL | 960 | μL |
| Primer mix | 5 | μL | 480 | μL |
| Total quantity | 15 | μL | 720 | μL |

2) The prepared PCR master mix was dispensed into a PCR tube.

3) 5 μL of DNA extracted from the sample or a positive control or negative control was added to the same PCR tube.

TABLE 1
Primer sequences for PCR amplification

| Name | Direction | Sequence |
|---|---|---|
| HSV1_F | Forward | GGAGTGCATAGTAGGTGA (SEQ ID NO: 27)/ |
| | | iSpC3/GATGATACGGTACATGGCC |
| | | (SEQ ID NO: 1) |
| HSV1_R | Reverse | /5Biosg/CTTGCGCATGACCATGT |
| | | (SEQ ID NO: 2) |
| HSV2_F | Forward | GTAACAACGGATAACTCTA (SEQ ID NO: 28)/ |
| | | iSpCS/ASAAATGATCCGATATATGGCT |
| | | (SEQ ID NO: 3) |
| HSV2_R | Reverse | CTCGTCCTCGTTGTGGA (SEQ ID NO: 4) |
| CT_F1 | Forward | AAGTGTGACCTGAAGATGT (SEQ ID NO: 29)/ |
| | | iSpC3/CTCCYTACATTGGAGTTAAATGG |
| | | (SEQ ID NO: 5) |
| CT_R1 | Reverse | CCTACTGCAATACCGCAAG |
| | | (SEQ ID NO: 6) |
| CT_F2 | Forward | AAGTGTGACCTGAAGATGT (SEQ ID NO: 30)/ |
| | | iSpC3/TAACTGTAGACTCGGCTTGG |
| | | (SEQ ID NO: 7) |
| CT_R2 | Reverse | /5Biosg/CCCTTTATACGCTCAAGCAATA |
| | | (SEQ ID NO: 8) |
| NG_F | Forward | CTAAGGCTAACTGACAATG (SEQ ID NO: 31)/ |
| | | CGGTCGAAGACAAAGACAAG |
| | | (SEQ ID NO: 9) |
| NG_R | Reverse | /5Biosg/GTTCATCCCACGCTTTAACT |
| | | (SEQ ID NO: 10) |
| TP_F | Forward | TCCACCAGATACCAAGTC (SEQ ID NO: 32)/ |
| | | iSpC3/TGCCGCGATATATTCCGC |
| | | (SEQ ID NO: 11) |
| TP_R | Reverse | GTTTGGATGCTTTTGGCCT |
| | | (SEQ ID NO: 12) |
| TV_F | Forward | ATCGTGAGTCATCCATAGT (SEQ ID NO: 33)/ |
| | | iSpC3/ATCAACGTCAAYTACACACTTC |
| | | (SEQ ID NO: 13) |
| TV_R | Reverse | /5Biosg/TAGAGATCCTTACGAACATCGA |
| | | (SEQ ID NO: 14) |
| UU_F | Forward | TACTGACTAACGAGACGAG (SEQ ID NO: 34)/ |
| | | iSpC3/CCTTTCAACAAAAGGGTATAGC |
| | | (SEQ ID NO: 15) |
| UU_R | Reverse | /5Biosg/GTATGTGGACATCCCCCA |
| | | (SEQ ID NO: 16) |
| UP_F | Forward | ATGTATCCCCAGACTGTAAC (SEQ ID NO: 35)/ |
| | | iSpC3/CACATTTTCACTTGTTTGAAGTG |
| | | (SEQ ID NO: 17) |
| UP_R | Reverse | /5Biosg/TCGTCCATAAGCAACTTTGC |
| | | (SEQ ID NO: 18) |
| MG_F | Forward | AACATCAGGCAATCGTAAG (SEQ ID NO: 36 / |
| | | iSpC3/GTTGACCATATGCAGTGTTATTTG |
| | | (SEQ ID NO: 19) |
| MG_R | Reverse | /5Biosg/TATCACCCATTGTTAGTGTTCC |
| | | (SEQ ID NO: 20) |
| MH_F | Forward | GCTGTTCGGTTTATGTCAC (SEQ ID NO: 37)/ |
| | | ACCAAAGATTACAAGATGCTCC |
| | | (SEQ ID NO: 21) |
| MH_R | Reverse | /5Biosg/CAGTTAAATCACGAATGAWCCAG |
| | | (SEQ ID NO: 22) |
| CA_F | Forward | TGAGATTTAGCGTAGATTT (SEQ ID NO: 38)/ |
| | | TTCTCCCTCAAACCGCTG |
| | | (SEQ ID NO: 23) |
| CA_R | Reverse | /5Biosg/CCGCAAGCAATGTTTTTGG |
| | | (SEQ ID NO: 24) |
| GV_F | Forward | GTAAGCAATAGCACCAAA (SEQ ID NO: 39)/ |
| | | iSpC3/GGATCAGATTGCAGCTACTG |
| | | (SEQ ID NO: 25) |
| GV_R | Reverse | /5Biosg/GAAGTCCAAATCAAGACCAAAC |
| | | (SEQ ID NO: 26) |

### 2) Polymerase Chain Reaction (PCR)

1) The above PCR tube was placed in the thermal cycler.

2) The thermal cycler was set up under the following conditions and the system was operated.

TABLE 2

| Step [symbol] | Temperature [symbol] | Time [symbol] | | Cycle [symbol] | [symbol] |
|---|---|---|---|---|---|
| UDG activation [symbol] | 50° C. [symbol] | 4 | min [symbol] | 1 [symbol] | [symbol] |
| Hot Taq Pol. activation [symbol] | 95° C. [symbol] | 10 | min [symbol] | 1 [symbol] | [symbol] |
| Denaturation [symbol] | 95° C. [symbol] | 15 | sec [symbol] | 30 [symbol] | [symbol] |
| Annealing/extension [symbol] | 70° C. [symbol] | 1 | min [symbol] | | [symbol] |
| Denaturation [symbol] | 95° C. [symbol] | 15 | sec [symbol] | 15 [symbol] | [symbol] |
| Annealing [symbol] | 58° C. [symbol] | 30 | sec [symbol] | | [symbol] |
| Extension [symbol] | 72° C. [symbol] | 30 | sec [symbol] | | [symbol] |
| Post extension [symbol] | 72° C. [symbol] | 1 | min [symbol] | 1 [symbol] | [symbol] |

### (3) Loading Into ULFA

1) After completion of the PCR reaction, 5 μL of the amplification product was loaded into the sample inlet of the ULFA device. One probe can detect one type of STI. For example, universal probe (Uprobe) 01 can detect CT.

TABLE 3
Universal probes

| Name [symbol] | No. [symbol] | Sequence [symbol] | STIs [symbol] |
|---|---|---|---|
| Uprobe 22 [symbol] | 40 [symbol] | TCACCTACTATGCACTCC [symbol] | HSV1 [symbol] |
| Uprobe 06 [symbol] | 41 [symbol] | TAGAGTTATCCGTTGTTAC [symbol] | HSV2 [symbol] |
| Uprobe 01 [symbol] | 42 [symbol] | ACATCTTCAGGTCACACTT [symbol] | CT [symbol] |
| Uprobe 11 [symbol] | 43 [symbol] | CATTGTCAGTTAGCCTTAG [symbol] | NG [symbol] |
| Uprobe 10 [symbol] | 44 [symbol] | GACTTGGTATCTGGTGGA [symbol] | TP [symbol] |
| Uprobe 07 [symbol] | 45 [symbol] | ACTATGGATGACTCACGAT [symbol] | TV [symbol] |
| Uprobe 08 [symbol] | 46 [symbol] | CTCGTCTCGTTAGTCAGTA [symbol] | UU [symbol] |
| Uprobe 09 [symbol] | 47 [symbol] | GTTACAGTCTGGGGATACAT [symbol] | UP [symbol] |
| Uprobe 04 [symbol] | 48 [symbol] | CTTACGATTGCCTGATGTT [symbol] | MG [symbol] |
| Uprobe 18 [symbol] | 49 [symbol] | GTGACATAAACCGAACAGC [symbol] | MH [symbol] |
| Uprobe 13 [symbol] | 50 [symbol] | AAATCTACGCTAAATCTCA [symbol] | CA [symbol] |
| Uprobe 03 [symbol] | 51 [symbol] | TTTGGTGCTATTGCTTAC [symbol] | GV [symbol] |
| Uprobe HC [symbol] | 52 [symbol] | CCTATTATGTATTGACGATG [symbol] | Internal control [symbol] |

2) 50 μL of running buffer was loaded into the sample inlet of the same ULFA device and the PCR amplification product was run for 10 minutes.

3) 50 μL of washing buffer was loaded into the sample inlet of the same ULFA device and run for 10 minutes to clean off the nitrocellulose membrane.

4) The amplification result was determined based on the presence or absence of a red line formed at the number indicated on the device (with line: positive, without line: negative).

### (4) Result Judgment

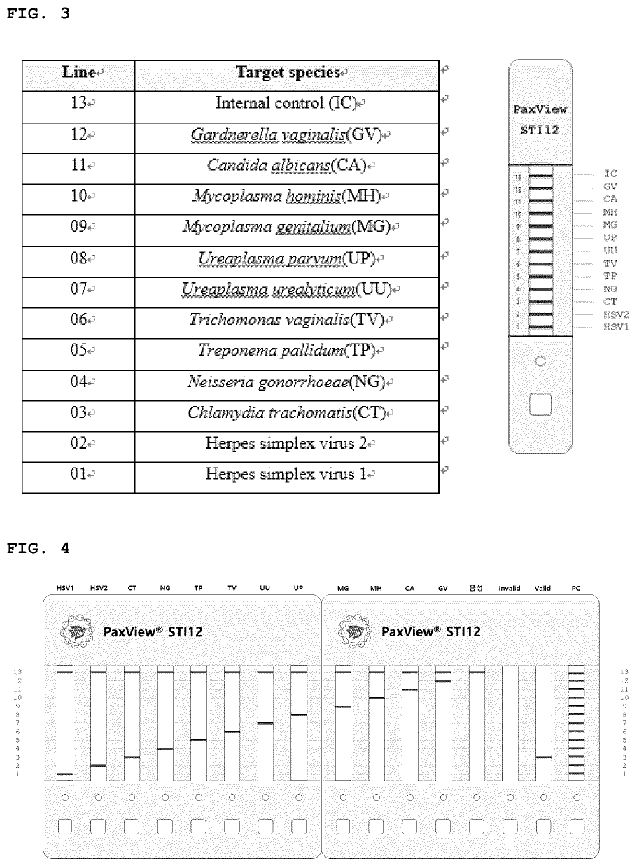

The band that reacted with the PCR amplification product in the ULFA device was visually checked to determine whether it was negative or positive (see FIGS. 3 and 5).

The technology applied to the PaxView STI12 MPCR-ULFA kit consists of one panel. In the technology, the PCR product produced by performing multiplex PCR for 12 types at the same time is detected in ULFA (universal lateral flow assay). Using 12 probes immobilized on the nitrocellulose membrane of the ULFA device, STI types specific to primers with a universal region containing a nucleic acid oligomer complementary to each probe can be detected. With one set of 12 types of universal probes (Uprobe), it is possible to detect all 12 types of STIs and an internal control.

### Industrial Applicability

The kit according to the present invention makes it possible to diagnose infection with 12 types of sexually transmitted infection-causing pathogens using only a trace amount of sample genes. In addition, by amplifying and diagnosing 12 types of sexually transmitted infection-causing pathogen-specific genes, the kit enables rapid and accurate qualitative molecular diagnosis of 12 types of sexually transmitted infection-causing pathogens. In addition, since the results are checked through a lateral flow assay after gene amplification, the kit is economical as it does not require expensive equipment like that used in real-time PCR. In addition, since the agarose gel preparation process for electrophoresis to confirm the amplified gene as in conventional PCR is not required, the kit can confirm results faster than the PCR-electrophoresis method.

Although the present invention has been described in detail with reference to specific features, it will be apparent to those skilled in the art that this description is only of a preferred embodiment thereof, and does not limit the scope of the present invention. Thus, the substantial scope of the present invention will be defined by the appended claims and equivalents thereto.

### Sequence Listing Free Text

Electronic file attached.

1. A kit for detecting sexually transmitted infection-causing pathogens, comprising:
(a) a primer containing: a complementary nucleic acid oligomer capable of binding to and amplifying a gene specific to a sexually transmitted infection-causing pathogen selected from the group consisting of Chlamydia trachomatis (CT), Neisseria gonorrhea (NG), Treponema pallidum (TP), Trichomonas vaginalis (TV), Ureaplasma urealyticum (UU), Ureaplasma parvum (UP), Mycoplasma genitalium (MG), Mycoplasma hominis (MH), Gardnerella vaginalis (GV), Candida albicans (CA), Herpes simplex virus 1 (HSV1), and Herpes simplex virus 2 (HSV2); and a nucleic acid oligomer non-complementary to the specific gene; and
(b) a probe that binds complementary to the non-complementary nucleic acid oligomer.

2. The kit according to claim 1, wherein the complementary nucleic acid oligomer of the primer (a) is selected from the group consisting of the sequences of SEQ ID NO: 1 to SEQ ID NO: 26.

3. The kit according to claim 1, wherein the complementary nucleic acid oligomer of the primer (a) comprises:
a complementary nucleic acid oligomer comprising the sequences of SEQ ID NO: 1 and SEQ ID NO: 2, which binds to the HSV1-specific gene;
a complementary nucleic acid oligomer comprising the sequences of SEQ ID NO: 3 and SEQ ID NO: 4, which binds to the HSV2-specific gene;
a complementary nucleic acid oligomer comprising a sequence selected from the group consisting of SEQ ID NO: 5 to SEQ ID NO: 8, which binds to the CT-specific gene;
a complementary nucleic acid oligomer the sequences of SEQ ID NO: 9 and SEQ ID NO: 10, which binds to the NG-specific gene;
a complementary nucleic acid oligomer the sequences of SEQ ID NO: 11 and SEQ ID NO: 12, which binds to the TP-specific gene;
a complementary nucleic acid oligomer comprising the sequences of SEQ ID NO: 13 and SEQ ID NO: 14, which binds to the TV-specific gene;
a complementary nucleic acid oligomer comprising the sequences of SEQ ID NO: 15 and SEQ ID NO: 16, which binds to the UU-specific gene;
a complementary nucleic acid oligomer comprising the sequences of SEQ ID NO: 17 and SEQ ID NO: 18, which binds to the UP-specific gene;
a complementary nucleic acid oligomer comprising the sequences of SEQ ID NO: 19 and SEQ ID NO: 20, which binds to the MG-specific gene;
a complementary nucleic acid oligomer comprising the sequences of SEQ ID NO: 21 and SEQ ID NO: 22, which binds to the MH-specific gene;
a complementary nucleic acid oligomer comprising the sequences of SEQ ID NO: 23 and SEQ ID NO: 24, which binds to the CA-specific gene; or
a complementary nucleic acid oligomer comprising the sequences of SEQ ID NO: 25 and SEQ ID NO: 26, which binds to the GV-specific gene.

4. The kit according to claim 1, wherein the non-complementary nucleic acid oligomer of the primer (a) is selected from the group consisting of the sequences of SEQ ID NOs: 27 to 39.

5. The kit according to claim 1, wherein the probe (b) is selected from the group consisting of the sequences of SEQ ID NOs: 40 to 52.

6. The kit according to claim 1, wherein, when a product amplified by the primer (a) is injected into a lateral side of a membrane, the amplified product moves and a complementary hybridization reaction between the probe immobilized on the membrane and the nucleic acid oligomer contained in the amplified product occurs.

7. A method of detecting sexually transmitted infection-causing pathogens in vitro using the kit according to any one of claims 1 to 6.

8. A method for detecting sexually transmitted infection-causing pathogens, comprising steps of:
(a) reacting a nucleic acid extracted from a sample with a primer containing: a complementary nucleic acid oligomer that binds to a gene specific to a sexually transmitted infection-causing pathogen selected from the group consisting of Chlamydia trachomatis (CT), Neisseria gonorrhea (NG), Treponema pallidum (TP), Trichomonas vaginalis (TV), Ureaplasma urealyticum (UU), Ureaplasma parvum (UP), Mycoplasma genitalium (MG), Mycoplasma hominis (MH), Gardnerella vaginalis (GV), Candida albicans (CA), Herpes simplex virus 1 (HSV1), and Herpes simplex virus 2 (HSV2); and a nucleic acid oligomer non-complementary to the gene specific to the sexually transmitted infection-causing pathogen; and
(b) reacting the product of the reaction with a probe that binds complementary to the non-complementary nucleic acid oligomer.

9. The method according to claim 8, wherein the complementary nucleic acid oligomer of the primer (a) is selected from the group consisting of the sequences of SEQ ID NO: 1 to SEQ ID NO: 26.

10. The method according to claim 9, wherein the complementary nucleic acid oligomer of the primer (a) comprises:
a complementary nucleic acid oligomer comprising the sequences of SEQ ID NO: 1 and SEQ ID NO: 2, which binds to the HSV1-specific gene;
a complementary nucleic acid oligomer comprising the sequences of SEQ ID NO: 3 and SEQ ID NO: 4, which binds to the HSV2-specific gene;
a complementary nucleic acid oligomer comprising a sequence selected from the group consisting of SEQ ID NO: 5 to SEQ ID NO: 8, which binds to the CT-specific gene;
a complementary nucleic acid oligomer the sequences of SEQ ID NO: 9 and SEQ ID NO: 10, which binds to the NG-specific gene;
a complementary nucleic acid oligomer the sequences of SEQ ID NO: 11 and SEQ ID NO: 12, which binds to the TP-specific gene;
a complementary nucleic acid oligomer comprising the sequences of SEQ ID NO: 13 and SEQ ID NO: 14, which binds to the TV-specific gene;
a complementary nucleic acid oligomer comprising the sequences of SEQ ID NO: 15 and SEQ ID NO: 16, which binds to the UU-specific gene;
a complementary nucleic acid oligomer comprising the sequences of SEQ ID NO: 17 and SEQ ID NO: 18, which binds to the UP-specific gene;
a complementary nucleic acid oligomer comprising the sequences of SEQ ID NO: 19 and SEQ ID NO: 20, which binds to the MG-specific gene;
a complementary nucleic acid oligomer comprising the sequences of SEQ ID NO: 21 and SEQ ID NO: 22, which binds to the MH-specific gene;
a complementary nucleic acid oligomer comprising the sequences of SEQ ID NO: 23 and SEQ ID NO: 24, which binds to the CA-specific gene; or a complementary nucleic acid oligomer comprising the sequences of SEQ ID NO: 25 and SEQ ID NO: 26, which binds to the GV-specific gene.

11. The method according to claim 8, wherein the non-complementary nucleic acid oligomer of the primer (a) is selected from the group consisting of the sequences of SEQ ID NOs: 27 to 39.

12. The method according to claim 8, wherein the probe (b) is selected from the group consisting of the sequences of SEQ ID NOs: 40 to 52.

13. The method according to claim 8, wherein, when a product amplified by the primer (a) is injected into a lateral side of a membrane, the amplified product moves and a complementary hybridization reaction between the probe immobilized on the membrane and the nucleic acid oligomer contained in the amplified product occurs.
